(19) United States
(12) Patent Application Publication
(10) Pub. No.: US 2008/0046012 A1
(43) Pub. Date: Feb. 21, 2008

(54) METHOD AND SYSTEM FOR MODULATING ENERGY EXPENDITURE AND NEUROTROPHIC FACTORS

(76) Inventors: Alejandro Covalin, Edina, MN (US); Jack W. Judy, Los Angeles, CA (US); Avi Feshali, Los Angeles, CA (US)

Correspondence Address: KLEIN, O'NEILL & SINGH, LLP, 43 CORPORATE PARK, SUITE 204, IRVINE, CA, 92606, US

(21) Appl. No.: 11/851,821

(22) Filed: Sep. 7, 2007

Related U.S. Application Data
(63) Continuation-in-part of application No. PCT/US06/09255, filed on Mar. 15, 2006.
(60) Provisional application No. 60/661,707, filed on Mar. 15, 2005.
(60) Provisional application No. 60/741,803, filed on Dec. 2, 2005.

(51) Int. Cl. A61N 1/00

(52) U.S. Cl. 607/2

(57) ABSTRACT

A method system for modulating the energy expenditure and/or the expressed brain-derived neurotrophic factor (BDNF) in the brain of an individual is performed by a system that includes a control device that generates a stimulation pattern from a predetermined set of stimulation parameters, and that converts the stimulation pattern into a stimulation signal. A stimulation signal delivery mechanism, configured for implantation into a selected part of the brain, receives the stimulation signal from the control device and delivers the signal to the selected part of the brain. The stimulation signal may be an electrical signal delivered by a brain-implantable electrode, or a chemical signal in the form of a drug dosage regimen delivered by an implantable micropump under the control of the control device. Modulation of the energy expenditure and/or BDNF is achieved by the stimulation of the hypothalamus, either directly or indirectly, by the stimulation signal.

### CROSS-REFERENCE TO RELATED APPLICATIONS

This application is a Continuation-in-Part of co-pending International Application No. PCT/US2006/009255, filed Mar. 15, 2006, the disclosure of which is incorporated herein by reference in its entirety. International Application No. PCT/US2006/009255 claims the benefit, under 35 U.S.C. § 119(e), of U.S. Provisional Patent Application No. 60/661,707, filed Mar. 15, 2005, and U.S. Provisional Patent Application No. 60/741,803, filed Dec. 2, 2005, the disclosures of which are incorporated herein by reference in their entirety.

### FEDERALLY SPONSORED RESEARCH OR DEVELOPMENT

Not Applicable.

### BACKGROUND OF THE INVENTION

Morbid obesity is second only to tobacco in causing the greatest number of deaths in the United States (i.e., annually causing 300,000 deaths as estimated for the year 2000) and has an estimated annual economic cost of $75 billion dollars. Obesity arises when the natural energy-homeostasis system is out of balance and can trigger a range of health-related problems, such as coronary heart disease, type-2 diabetes, hypertension, stroke, certain types of cancer, musculoskeletal disorders, gallbladder disease, and high blood cholesterol.

To treat morbid obesity, individuals typically use either a pharmacological and/or a surgical approach. The pharmacological approach promotes drugs that suppress appetite and/or prevent fat from being absorbed, while the surgical approach aims to either reduce stomach size (restrictive surgery) or decrease food absorption (malabsorbtive surgery). Since the pharmacological approach affects the whole body, it can cause some serious side effects (e.g., uncontrollably increasing heart rate and both diastolic and systolic pressure). The surgical approaches are not only costly, but also risky. Two percent of patients who take the surgical approach die and 20% of the patients have to be readmitted to the hospital during the first year after surgery. In addition, post-surgical patients must completely change their eating habits to maintain body weight.

Obesity is an energy imbalance in which the average energy expenditure of an individual is lower than his/her energy intake (i.e., calories from food intake). The energy-homeostasis system in the human body creates an energy equilibrium (i.e., energy in=energy out) in the body, to control body weight (BW). However, psychological, pathological, and social factors can force an energy imbalance, generating body-weight fluctuations that depend on the long-term ratio of food intake (FIN) and the total energy expenditure (TEE) of the individual.

The physiological control of both energy expenditure and energy intake is highly dependent on the neuronal activity in the hypothalamus of the brain. The hypothalamus monitors various molecules (e.g., leptin, insulin and glucose) to determine the energy availability and to accordingly modify the energy expenditure. Experimental data have shown that the energy expenditure can be artificially modulated by stimulating the hypothalamus, in particular the hypothalamic area called the ventromedial hypothalamic nucleus (VMH). Energy expenditure can be increased or decreased depending on the stimulating parameters. Also, depending on the stimulating parameters, an increase in energy expenditure can trigger, among other things, a fat breakdown (lipolysis) which in turn leads to a reduction in appetite. In such a case, the body weight is reduced by the cumulative effects of both the increase in energy expenditure and the reduction of appetite.

Obsesity problems may also be overcome by deep brain stimulation, wherein electrical stimulation, chemical stimulation, or a combination of electrical and chemical stimulation, modulates the food intake. Prior methods and systems have suggested that the use of electrical stimulation, chemical stimulation, or a combination of electrical and chemical stimulation in the hypothalamus may be able to modify the energy intake (i.e., food intake). However, the prior art does not provide any method of addressing obesity by modulating the energy expenditure.

Also, when electrical stimulation is applied, it is the magnitude of the electrical current injected, and not the applied voltage, that drives the modulation of neuronal activity. Furthermore, the charge injection must be balanced (i.e. have a mathematical mean equal to zero) in order to prevent a lesion. The prior art does not use a charge-balanced protocol, a requisite in order to avoid a lesion on the brain. The prior art uses voltage to control the electrical stimulation (voltage control) and not current (current control), despite the disadvantages of voltage control. With current control, the stimulation is steady throughout the pulse, while with voltage control, stimulation is highest only at the beginning of the pulse. Additionally, the stimulation efficacy using current control remains constant even when the impedance of the electrode(s) increases due to tissue build-up around the electrode(s). In contrast, stimulation efficacy when using voltage control drops as the electrode impedance increases due to such tissue build-up.

In addition, studies have been done on the effects of brain-derived neurotrophic factor (BDNF). It has been established, for example, that BDNF, a naturally-occurring molecule in the brain, as explained below, has been shown to have marked neuroprotective and neuroregenerative effects. Diseases characterized by neurological damage, such as Alzheimer's and Parkinson's, affect millions of persons. Increasing, in a controlled manner, the concentration or levels of BDNF in certain areas of the brain may prove to be an effective therapy for at least some of these neurological conditions.

Thus, a system and a method of stimulating the brain to modulate both BDNF levels and energy expenditure would provide significant benefits in the treatment of a wide variety of diseases and conditions.

### SUMMARY OF THE INVENTION

In accordance with the present invention, changes in the energy expenditure of a subject are achieved by electrically or chemically stimulating a particular region in the subject's hypothalamus (i.e., the ventromedial hypothalamic nucleus or VMH). The invention can also be implemented by chemical stimulation/inhibition through the delivery of appropriate dosages of suitable chemicals into the cerebral ventricles, the delivery of which can be effected either directly (e.g., by injecting the substances into the cerebral ventricles) or indirectly (e.g., by injection into the cerebrospinal fluid, e.g., in the cervical spinal chord). The invention can also be carried out by electrically or chemically stimulating/inhibiting the sympathetic nervous system, such as at the celiac ganglion or at its afferents or efferent fibers (e.g. at the efferent fibers enervating the adrenal medulla).

Stimulation of the hypothalamus, particularly the dorsomedial portion of the ventromedial hypothalamic nucleus (dmVMH) has several effects:

1. Energy expenditure is directly modulated via sympathetic activation, partially by activating the hypothalamic-splanchnic pathway.

2. Lypolysis (break-down of fat) occurs when energy expenditure is increased via an increase in sympathetic activity.

3. Glucose is released into the blood when energy expenditure is increased via an increase in sympathetic activity.

4. Food intake is indirectly affected by dmVMH stimulation due to changes in the glucose concentration in the blood resulting from the stimulation. For example, if energy expenditure is increased via sympathetic activation, then more glucose is released into the blood circulation. Blood glucose is both directly and indirectly sensed by several hypothalamic nuclei. In particular, when blood glucose increases, the lateral hypothalamic area (LHA), which is partially responsible for initiating a feeding response, suppresses the drive to eat, thereby effectively decreasing food intake.

When using electrical stimulation, in order to prevent tissue damage, the net amount of electrical charge delivered must be zero. The stimulation amplitude has to be kept low to avoid damaging the tissue and/or the electrodes. The actual amplitude will vary from case to case (depending on the relative position of the electrode within the brain). The range of the stimulation frequency depends on the desired outcome. In electrical stimulation directed to the VMH, it has been determined that signals having frequencies ranging from 25 to 100 Hz increase the resting energy expenditure, while high frequencies (e.g., 7 KHz) produce a decrease in the resting energy expenditure. The electrical signal is delivered as a rectangular current-pulse signal. The specific frequency at which optimum results are obtained, in terms of increasing resting energy expenditure, will, of course, vary from subject to subject.

Chemical stimulation can be chronically or acutely delivered via an implanted catheter or a simple injection. The implanted catheter can be supplied via an implanted pump and reservoir. The chemicals can be delivered directly or indirectly into the hypothalamus or into the cerebral ventricles (e.g., into the third ventricle). Due to the fact that the blood-brain-barrier is permeable at the median eminence, an indirect way to deliver the chemicals into the hypothalamus is by introducing them into the blood circulation. Releasing the chemicals into the third ventricle has the same qualitative effect as releasing them into the hypothalamus. Also, since cerebrospinal fluid is re-circulated, an indirect way to introduce at least some of the administered dosage of the chemical into the cerebral ventricles is by releasing the chemical into the cerebrospinal fluid, for example, in the cervical spinal chord. Releasing the chemical into the cerebrospinal fluid outside of the brain has the further advantage of stimulating some targets in the medulla and the spinal chord. For example, stimulating the melanocortin receptors, particularly the MC4 receptors, in the medulla and the spinal chord will increase the energy expenditure via sympathetic activation.

Some of the chemicals that can be used when targeting the hypothalamus or the cerebrospinal fluid are agonists and antagonists of receptors for orexin (OX1R and OX2R), neuropeptide Y (NPY), melanocortin (MC3R and MC4R), leptin and gherelin.

Chemical or electrical stimulation of the sympathetic nervous system can be achieved in a similar manner to the methods described above for the central nervous system (CNS). The main difference for the electrical protocol is that a different electrode is needed and that the stimulation amplitudes might be different. The main difference in the chemical protocol is that the stimulating/inhibiting substances are different from those used in the CNS. For example, if the modulation is done at the ganglia, then an agonist or an antagonist (depending on the desired response) of the acetylcholine receptor should be used. If the modulation is done at a postganglionic target, then an agonist or an antagonist of the norepinephrine receptor should be used.

A particular advantage of the present invention is its ability to modulate brain-derived neurotrophic factor (BDNF), which is a molecule that, aside from playing an important role in the memory and learning process, also possesses neuroprotective and neuroregenerative properties. For example, higher levels of BDNF in the hippocampus have been associated with increased neurocognitive performance, while lower BDNF levels in particular brain regions have been associated with certain neurodegenerative diseases, such as Alzheimer's (low hippocampal BDNF) and Parkinson's (low BDNF in the Substantia Nigra). Since BDNF protects neurons from dying, the low levels of BDNF in these regions results in decreased neuron survival, which, in turn, contributes to the progression of these neurological diseases. Consequently, a therapy capable of increasing BDNF may ameliorate the symptoms of these diseases or even reverse the neurological damage they effect. In addition to promoting neuronal survival and enhancing neuronal plasticity, BDNF plays an important role in the control of the energy homeostasis system.

It has been discovered that stimulation of the hypothalamus can modulate the expression of BDNF, particularly in the hippocampus, but also in other regions of the brain. In particular, by electrical stimulation of the hypothalamus, BDNF mRNA (messenger RNA) in the hippocampus can be modulated (increased or decreased), depending on the frequency of the stimulation signal. The hippocampus is a brain region that is intimately related to the memory and learning processes. It has also been shown that a higher cognitive performance correlates with higher concentrations of BDNF in the hippocampus. In accordance with the present invention, stimulation of the VMH at frequencies between 25 Hz and 100 Hz triggers an increase in hippocampal BDNF mRNA. In experiments with rats, for example, a stimulation frequency of 50 Hz yielded a 66%±14% increase in hippocampal BDNF mRNA. Conversely, stimulation of rats at 7 KHz showed a decrease in hippocampal BDNF mRNA by 33%±8%.

The invention may be carried out, in one embodiment, by implanting an electrode into the hypothalamus (in particular into the VMH) and connecting the electrode to an implanted container or box containing all the electronics required to generate and control the electrical stimulation. The electronics may advantageously be powered with a rechargeable battery, which may be recharged via induction using an external inductive recharging device. By setting the amount of time per hour that the stimulator is ON (i.e., by setting the duty cycle), the average increase in energy expenditure and the average decrease in food intake can be controlled.

The present invention is advantageous in that it modulates the brain's regulation of energy expenditure and food intake, while also modulating the brain's expression of a biological factor (BDNF) that promotes and enhances the protection and regeneration of neural cells, and that facilitates processes that are needed in memory and learning. Furthermore, deep brain stimulation in the hypothalamus, in accordance with the present invention, can be used to increase, in a controlled and reversible manner, the average energy expenditure and food intake, as well as the BDNF concentration in several regions of the brain. In the case of obesity, for example, the present invention offers an alternative to surgical options that are not reversible, cannot be controlled, and are relatively risky.

The present invention is a system and a method for stimulating the hypothalamus for modulating the energy expenditure and/or the BDNF expression of an individual. Electrical and/or chemical stimulation (local drug delivery) can be delivered (directly or indirectly) into the hypothalamus to modify the hypothalamic neuronal activity of the individual. For electrical stimulation, a stimulation pattern is generated by a control device (e.g., a microcontroller, microprocessor, state machine, or other suitable electronic device or circuit). The stimulation pattern is then converted into a stimulation current signal, and delivered to the hypothalamus via an implanted electrode(s). For chemical stimulation, a control device (e.g. a microcontroller, microprocessor, state machine, or other suitable electronic device or circuit) controls a micropump that delivers a dose of a stimulating chemical from a reservoir into the hypothalamus, into a cerebral ventricle, into the cerebrospinal fluid, or into the afferents/efferents of the celiac ganglia, via a an implanted conduit, such as a catheter. A sensor (which may be one or more of the electrodes functioning as a sensor, a separate implanted sensor, or a non-invasive indirect sensing device) may optionally be used to provide a feedback signal to the control device to automatically adjust the stimulation parameters. The system and method may include either electrical or chemical stimulation alone, or a combination of both types of stimulation.

In a first broad aspect, the present invention is a method for stimulating the hypothalamus for modulating the BDNF expression and/or the energy expenditure and food intake of a subject having a brain, wherein the method comprises the steps of (1) generating a stimulation pattern with a control device (such as a microprocessor, microcontroller, state machine, or other suitable electronic device or circuit) from a predetermined set of stimulation parameters; (2) converting the stimulation pattern into a stimulation signal; and (3) delivering the stimulation signal to a selected part of the brain to stimulate the hypothalamus. The method may additionally comprise the steps of (4) generating a feedback signal from a sensor, wherein the feedback signal represents the value of a measured parameter; and (5) adjusting the stimulation parameters in response to the feedback signal. In a first specific embodiment, the stimulation signal is an electrical signal delivered to the hypothalamus or to the VMH-splanchnic pathway (e.g., to the afferents/efferents of the celiac ganglia) by an implanted electrode. In a second specific embodiment, the stimulation signal is a chemical signal delivered to the hypothalamus by means of a dosage regimen of an appropriate chemical. The chemical can be delivered either directly to the hypothalamus, or indirectly via a cerebral ventricle, the cerebrospinal fluid or the blood circulation, and it can be delivered through an implanted conduit or catheter, through a transcutaneous port, or by injection. In a third specific embodiment, the stimulation signal is a combination of an electrical signal and a chemical signal, respectively delivered as described above.

In a specific example of the electrical stimulation embodiment, the method includes implanting a stimulating/sensing electrode assembly into the brain; generating a stimulation pattern with a control device (such as a microprocessor, microcontroller, state machine, or other suitable electronic device or circuit) from a set of stimulation parameters; and converting the stimulation pattern into an electrical stimulation signal; delivering the electrical stimulation signal to the hypothalamus or the VMF-splanchnic pathway via the implanted electrode assembly to stimulate the hypothalamus so as to modulate energy expenditure and food intake and/or the BDNF level expressed in the certain parts of the brain, particularly the hippocampus. The method may also include the step of adjusting the stimulation parameters based on a feedback signal from a sensor that may be at least one sensing electrode in the implanted electrode assembly. Alternatively, the electrode assembly may include one or more electrodes that perform only a stimulation function, and the feedback signal may be generated by a separate implanted sensor or by a non-invasive sensing device.

In a specific example of the chemical stimulation embodiment, the method includes implanting a drug delivery mechanism in the brain; generating a stimulation pattern with a control device (such as microprocessor, microcontroller, state machine, or other suitable electronic device or circuit) from a set of stimulation parameters; converting the stimulation pattern into a control signal; delivering the control signal to the drug deliver mechanism that responds to the control signal by generating a stimulation signal in the form of a drug dosage regimen that is delivered to the hypothalamus (directly or indirectly, as explained above) to stimulate the hypothalamus; and (optionally) adjusting the stimulation parameters based on a feedback signal from a sensor.

It is understood that a third embodiment of the method according to the invention may comprise a combination of the electrical and chemical stimulation embodiments.

In another broad aspect, the present invention is a system for stimulating the hypothalamus for modulating the BDNF expression and/or the energy expenditure of a subject having a brain, the system comprising a microcontroller (or equivalent control device) programmed or operated to generate a stimulation pattern from a predetermined set of stimulation parameters, and to convert the stimulation pattern into a stimulation signal; and a stimulation signal delivery mechanism, configured for implantation into a selected part of the brain, that receives the stimulation signal from the control device and delivers the stimulation signal to the selected part of the brain. The system may also include a sensor that generates a feedback signal in response to measured parameters affected by the stimulation signal, whereby the control device is programmed or operated to receive the feedback signal and to adjust the stimulation parameters in response thereto. The sensor may be a sensing electrode in an implanted electrode assembly, a separate implanted sensor, or a non-invasive sensing device. In a first specific embodiment, the stimulation signal is an electrical signal delivered to the hypothalamus or the VMH-splanchnic pathway by at least one stimulating electrode in an implanted electrode assembly. In a second specific embodiment, the stimulation signal is a chemical signal delivered to the hypothalamus either directly or indirectly by any of the fluid delivery means mentioned above.

In a specific example of the electrical stimulation embodiment, the stimulating signal delivery mechanism includes at least one stimulating electrode in the implantable electrode assembly. The stimulation signal is an electrical stimulation signal, preferably, but not necessarily, a controlled current signal. The electrical stimulation signal is delivered to the selected part of the brain via the stimulating electrode. In a specific example of the chemical stimulation embodiment, the stimulation signal delivery mechanism comprises an implantable micropump operated under the control of the control device. The stimulation signal is in the form of a drug dosage regimen delivered directly or indirectly to the hypothalamus by the micropump in response to a control signal generated by the control device.

It is understood that a third embodiment of the system according to the invention may comprise a combination of the electrical and chemical stimulation embodiments described above.

This brief summary has been provided so that the nature of the invention may be understood quickly. A more complete understanding of the invention can be obtained by reference to the following detailed description of the preferred embodiments thereof in connection with the attached drawings.

### BRIEF DESCRIPTION OF THE DRAWINGS

The foregoing features and other features of the present invention will now be described with reference to the drawings of a preferred embodiment. In the drawings, the same components have the same reference numerals. The illustrated embodiment is intended to illustrate, but not to limit the invention. The drawings include the following Figures:

FIG. 1 is a diagrammatic representation illustrating the interaction among the different nuclei of the energy-homeostatis system;

FIG. 2A is an idealized view of an implantable electrode assembly of the type employed in the present invention;

FIG. 2B is a cross-sectional view taken along line B-B of FIG. 2A;

FIG. 2C is an idealized view of a modified version of the implantable electrode assembly;

FIG. 3 is a schematic diagram of a system for modulating the BDNF expression and/or energy expenditure of an individual, in accordance with the present invention;

FIG. 4 is a schematic diagram illustrating an active feedback circuit that automatically balances the injected and extracted charge to avoid damage to the tissue and to the electrode according to one aspect of the present invention;

FIG. 5 is a graph illustrating a biphasic stimulation waveform where the charge is automatically balanced using the active feedback circuit of FIG. 4;

FIG. 6 is a graph that illustrates the effect of stimulation frequency on nMEE;

FIG. 7 is a graph that illustrates the effect of stimulation frequency on hippocampal BDNF mRNA;

FIG. 8 is a graph that illustrates the effect of stimulation frequency on hippocampal NT3 mRNA;

FIG. 9 is a graph that illustrates a regression analysis performed between the hippocampal BDNF mRNA and the nMEE with all of the experimental data;

FIG. 10 is a graph that illustrates a regression analysis performed between the hippocampal BDNF mRNA and the nMEE with all of the experimental data except that performed at a stimulation signal frequency of 50 Hz;

FIG. 11 is a graph that illustrates the threshold to elicit an escape-response as a function of frequency;

FIG. 12 is a graph that illustrates the VMH stimulation effect on the TEE in the form of power; and

FIG. 13 is a bar graph that illustrates the VMH stimulation effect on the TEE in the form of cumulative energy.

### DETAILED DESCRIPTION OF THE INVENTION

The following detailed description is of the best currently contemplated mode of carrying out the invention. The description is not to be taken in a limiting sense, but is made merely for the purpose of illustrating the general principles of the invention.

The present invention is a system and method for stimulating the hypothalamus for modulating the expression of BDNF and/or the energy expenditure of an individual. In accordance with one embodiment of the invention an electrode assembly is implanted into the hypothalamus of the brain. Although the hypothalamus makes up only 0.4 percent of the brain tissue, it is an indispensable structure responsible for homeostatic processes, such as body-temperature regulation, diurnal/nocturnal rhythms, hydration, body weight, and food intake.

The hypothalamus has four regions along the anterior-posterior axis: (1) the preoptic region, (2) the chiasmatic region, (3) the tuberal region and, (4) the mammillary region. The preoptic region is comprised of the periventricular nuclei, the medial nuclei and the lateral preoptic nuclei. The medial nuclei and lateral preoptic nuclei contain temperature-sensing cells that are involved in the thermoregulation process and connect to other areas of the hypothalamus. The chiasmatic region is comprised of the suprachiasmatic (SCH) nuclei that regulates the individual's internal clock (circadian rhythm), the supraoptic (SON) nuclei, the paraventricular (PVN) nuclei that strongly influences food intake (FIN) by interacting with other hypothalamic nuclei (i.e., dorsomedial hypothalamic nucleus and lateral hypothalamic area), and the anterior hypothalamic (AHN) nuclei that integrate signals from other hypothalamic nuclei (i.e., the medial preoptic area and the ventromedial hypothalamic nucleus) eliciting defensive behaviors.

The tuberal region is comprised of the arcuate nucleus (ARC), the ventomedial hypothalamic nucleus (VMH), and the dorsomedial hypothalamic nucleus (DMH). The ARC possesses many intra-hypothalamic connections and is a control center that drives energy-conserving and energy-expending cascades. Different portions of the VMH regulate ovulation, aggression and energy expenditure. Also, the VMH appears to be the link by which the nutritional status gets integrated into circadian neuroendocrine responses. The DMH, which is connected with multiple hypothalamic nuclei, modulates insulin secretion as well as some autonomic functions (via the PVN) such as heart and respiration rate. The DMH and the PVN work as a functional unit modulating FIN.

The ARC receives information from both circulating molecules, due to a leaky blood-brain-barrier in the area, and direct neuronal inputs. The ARC can be considered to be both an integrative and a command center for the energy homeostasis system. In particular, signaling-molecules in the blood circulation are monitored through which long (leptin), middle (insulin) and short-term (glucose and gherelin) energy availability can be sensed. In normal circumstances, leptin, which is produced by the adipose tissue, circulates in the blood stream in a concentration that is proportional to the amount of total body-fat tissue. The concentration of gherelin, a hormone produced in the epithelial cells in the stomach, is at its lowest point after a meal, at which time it begins its ascent until the next meal.

The ARC receives neuronal inputs from regions inside and outside the hypothalamus. Its intra-hypothalamic afferents originate mainly at the PVN, and at the LHA. Most of its extra-hypothalamic afferents originate at the NTS, the amygdala, and the bed nucleus of the striaterminalis. The ARC contains at least two different neuronal populations that produce functionally antagonistic signaling molecules. One population produces pro-energy-conserving signaling molecules (ECm) and the other population produces pro-energy-expending signaling molecules (EEm). To regulate both FIN and the energy expended due to non-movement-related activities, these signaling molecules influence neuronal activity in other hypothalamic nuclei and in the ARC. Thus, the neuronal activity in the ARC tends to balance the energy expenditure (EE) and the FIN. The ARC monitors the energy status in the body and acts upon other hypothalamic nuclei in order to compensate for an imbalance in the energy system.

The PVN receives inputs from and sends outputs to most hypothalamic nuclei involved in the energy-homeostasis system. It also projects to both sympathetic and parasympathetic neurons functioning as a major integrating, processing, and actuating center for the energy-homeostatic system.

The VMH is anatomically divided and these divisions are likely to be functionally different. With respect to the energy-homeostasis system, the VMH integrates information about short-term and long-term energy availability, and it has functional connections from and to most of the other hypothalamic nuclei involved in the energy-homeostasis system. VMH activity influences, FIN, EE, lipolysis, and glucose uptake in muscles.

The DMH constitutes an integrative center for intra and extra hypothalamic inputs that modulate aspects of the energy-homeostasis system, mainly by influencing PVN activity.

The LHA receives information from many systems including the gastro-intestinal (GI) tract. The LHA integrates information from all of these systems, and in turn it influences the expression of ECm and EEm in the ARC as well as the glucose sensitivity in the VMH.

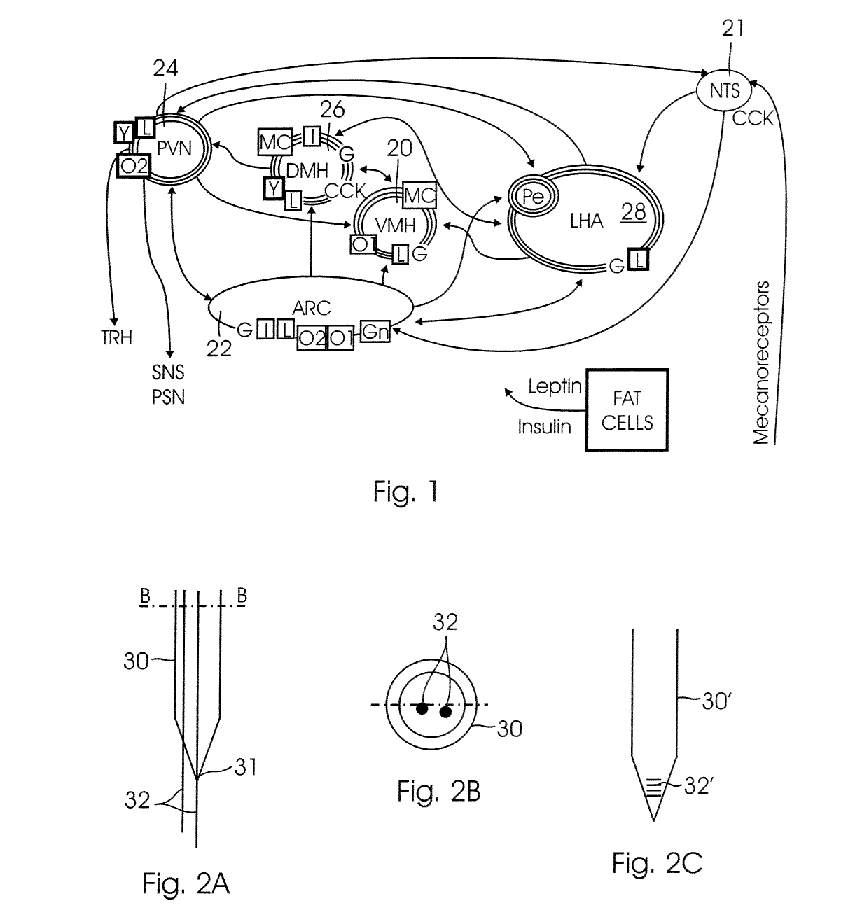

Turning to FIG. 1, an energy-homeostasis system of an individual is shown. The energy-homeostasis system includes both hypothalamic and extra-hypothalamic centers that are involved in processes regulating both the energy intake (EIN) and the Total Energy Expenditure “TEE”. While EIN has one component (food intake or FIN), TEE can be divided into two main components: the energy expended due to movement-related activities (called mechanical energy expenditure, or “MEE”) and the energy expended due to non-movement-related activities (called non-mechanical energy expenditure, or “nMEE”). This division is such that at any given time the sum of these two components is equal to the TEE. In humans, the nMEE represents up to 70% of the TEE. Body weight that remains relatively constant is due to the proper regulation of the nMEE.

The energy-homeostasis system is controlled by the neuronal activity in the hypothalamus. However, psychological, pathological, and social factors can force the energy equation (energy in=energy out) out of balance, generating body weight fluctuations that depend on the long-term ratio of FIN and the TEE.

Several mutually interacting hypothalamic regions control the FIN and the TEE. The Arcuate Nucleus (ARC) 22, the Paraventricular nucleus (PVN) 24, the Ventromedial Hypothalamic Nucleus (VMH) 20, the Dorsomedial Hypothalamic Nucleus (DMH) 26 and the Lateral Hypothalamic Area (LHA) 28 play a vital role in regulation of FIN and TEE. VMH 20 directly affects the energy expenditure, which in turn indirectly affects the FIN. Electrically stimulating the VMH 20 increases the nMEE which is equal to TEE minus the mechanical energy expenditure (MEE) Indirectly, the VMH-stimulation-related nMEE increase produces a decrease in the FIN. Inhibiting VMH 20 activity by means of a lesion causes the exact opposite effects. The nMEE is increased by an increase of sympathetic activity, which is supported via lipolysis.

At least five hypothalamic nuclei are involved in the regulation of the FIN and the nMEE, as is shown in FIG. 1. These nuclei are ARC 2, PVN 24, VMH 20, DMH 26 and LHA 28. In addition, at least part of the nMEE regulation is exerted via sympathetic and parasympathetic modulation. Indirect connections between hypothalamic nuclei and the vagus nerve via the nucleus of the solitary tract (NTS) 21 provide signals that influence the FIN.

In accordance with one specific embodiment of the present invention, a particular region of the hypothalamus is electrically stimulated by at least one stimulation electrode in an implantable electrode assembly that is implanted in the hypothalamus, and particularly the VMH. The electrode assembly may be of the type shown in FIGS. 2A, 2B, and 2C, wherein the electrode assembly comprises an implantable conduit 30 having a distal tip 31 and containing at least one electrode 32 that extends distally from the tip 31. Alternatively, the one or more electrodes 32 may terminate flush with the tip 31, or may be exposed along the side of the conduit 30. The conduit 30 is made out of a biocompatible material (e.g., silicone, silicon, titanium, ceramic, etc.), and it can act as a mechanical substrate for implanting the electrodes 32 used for stimulations and/or sensing. Alternatively, the conduit 30 may serve as a fluid channel for chemical stimulation, or as both a substrate for the electrodes 32 and a fluid channel. FIGS. 2A and 2B show two electrodes 32 contained in the conduit 30, although this number is merely exemplary, and a single electrode, or more than two electrodes, may be used. Indeed, if the conduit is used only as a fluid channel in a chemical stimulation embodiment, the electrodes 32 may be absent. FIG. 2C illustrates a modified version of the conduit 30′ having electrodes 3′ incorporated onto its outer surface. The electrodes 32, 32′ are made of any suitable biocompatible conductive material (e.g. platinum, platinum-iridium, iridium, activated iridium oxide, titanium nitride, etc.).

Advantageously, one or more of the electrodes 32, 32′ can be selectively operated in either a sensing (recording) mode or a stimulation mode. In the recording mode, the electrodes act as sensors, recording data from the brain, while in the stimulation mode they stimulate that area of the brain in which the electrodes are implanted. The recording and stimulating functions can be performed with the same electrode, or with different electrodes.

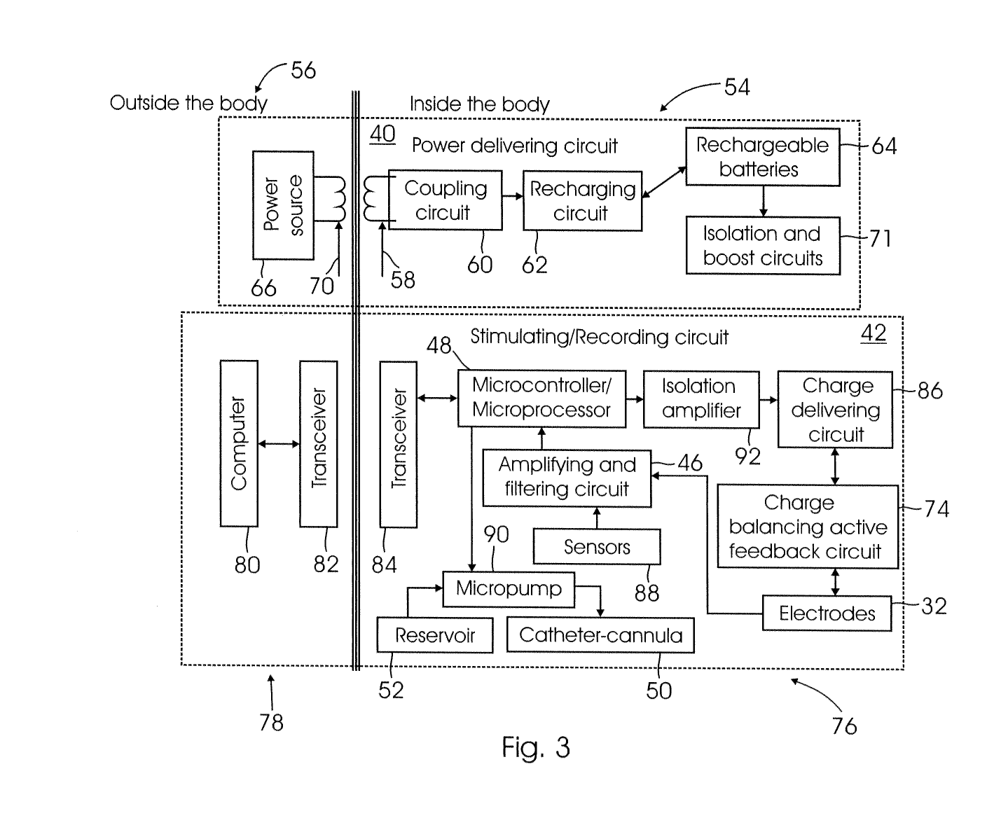

FIG. 3 is a schematic diagram of a system for hypothalamic stimulation for modulating the energy expenditure and/or BDNF expression of an individual, in accordance with the present invention. In this figure, and in the description that follows, the system includes means for both electrical and chemical stimulation. It will be understood that a system can be constructed in accordance with present invention that includes only electrical stimulation or only chemical stimulation. Furthermore, this exemplary system includes an implantable sensor, which may be one of the electrodes in an implantable electrode assembly. It is understood that a sensing function is optional, and may be performed by a separate, non-invasive sensing device. Finally, the system described below provides an electrical stimulation signal that is a controlled current signal, which is preferred over a controlled voltage signal for the reasons discussed above. It will be understood, however, that the stimulation signal may be a controlled voltage signal, and the modifications necessary to provide such a signal will readily suggest themselves to those skilled in the pertinent arts.

As shown in FIG. 3, a power delivering circuit 40 provides power to a stimulating/recording circuit 42 which includes the electrode assembly comprising one or more electrodes 32. When the stimulating/recording circuit 42 is in the simulation mode for electrical stimulation, at least one of the electrodes 32 (as described above with reference to FIGS. 2A-2C) carries an electrical stimulation signal from the power delivering circuit 40 into the neural tissue and back to the power delivering circuit 40. If stimulation is by means of a chemical stimulation signal, as described more fully below, an implantable catheter 50 may be provided, with a port for refilling from a chemical reservoir 52 though the skin.

In the recording mode, at least one of the electrodes 32 detects minute changes in the electrical potential in the neural tissue, which changes effectively convey the neuronal activity in the area. These minute changes are then delivered to an amplifying and filtering circuit 46 which filters and amplifies the signals before delivering them to a microcontroller 48 or equivalent control device, such as a microprocessor, state machine, or other functionally equivalent electronic device or circuit.

The power delivering circuit 40 includes an implantable portion 54 and a non-implantable external portion 56. The implantable portion 54 includes a battery power supply that may advantageously employ rechargeable batteries 64 as the power source. If rechargeable batteries are used, the implantable portion 54 would include an implanted inductor 58, a coupling circuit 60, and a recharging circuit 62, while the external portion 56 would include a power supply and coupling circuit 66 and an exterior inductor 70. By aligning and putting the exterior inductor 70 in close proximity to the implanted inductor 58, the batteries 64 can be recharged.

In the electrical stimulation embodiment of the present invention, isolation and boost circuits 71 can be used to isolate a charge delivering circuit 86 and a charge-balancing active-feedback circuit 74 from the rest of the stimulation/recording circuit 42. The stimulating/recording circuit 42 also has an external portion 78 and an implantable portion 76. The external portion 78 of the stimulating/recording circuit 42 includes a computer 80 and an external transceiver 82. The implantable portion 76 of the stimulating/recording circuit 42 includes an implanted transceiver 84, a microcontroller or equivalent control device 48, a charge-delivering circuit 86 (which includes voltage-current conversion circuitry) that receives a control signal from the control device 48 through an isolation amplifier 92, a charge-balancing active-feedback circuit 74, an amplifying and filtering circuit 46, at least one stimulating and/or recording electrode 32, and a sensor 88 (which may be an electrode 32 functioning in a sensing or recording mode). If only chemical stimulation is to be employed, the charge delivering circuit 86, the isolation amplifier 92, and the charge-balancing circuit 74 may be omitted. If chemical stimulation is used, with or without electrical stimulation, the system also includes the catheter 50, a micropump 90 and the reservoir 52. In the chemical stimulation embodiment, the electrodes 32 may be omitted, or, alternatively, at least one implanted electrode may be employed as a sensor. That is, the sensor 88 may be in the form of an implanted electrode. Furthermore, where chemical stimulation is employed, the conduit 30, described above, may be used to deliver the stimulation chemical in place of the catheter 50.

The control device 48 can be used in either an opened-loop or a closed-loop mode. In the opened-loop mode, the stimulation is performed without taking into account the information received from the sensor 88. In the closed-loop mode, the stimulation is performed and controlled at least partially by the information received from the sensor 88.

The control device 48 controls the stimulation parameters. In the electrical stimulation embodiment, these parameters may include electrical current intensity, pulse width, pulse frequency, the wave shape, the duration of stimulation (i.e., how long the stimulation is delivered each time it is turned on) and the repetition rate of stimulation (i.e. how often is the stimulation turned on). In the chemical stimulation embodiment, the control device 48 controls the local drug delivery stimulation parameters, including the drug type, the flow rate, the total volume per stimulation session, and the repetition rate (i.e., how often the stimulation session occurs). The stimulation parameters may optionally be adjustable, e.g., by wireless communication between the external computer 80 and the internal (implanted) control device 48 via the external and implanted transceivers 82, 84. Alternatively, a fixed set of stimulation parameters can be employed.

The trajectory of the electrode(s) 32 and the conduit 30, as well as the location of the implanted device, is determined for each individual on a case by case basis. The implantation of the electrode(s) 32 and the conduit 30 may be performed using a neurosurgical technique known as stereotactic neurosurgery. Typically, the electrodes and/or the conduit are implanted in the VMH, while the sealed biocompatible container or box (not shown) containing the electronics and/or the micropump and reservoir (described below) can be implanted in any other part of the body preferred by the surgeon. If a discrete sensor 88 is employed, it may also be implanted into the VMH using the same neurosurgical technique, or it may be implanted elsewhere in the brain, or in another part of the body, depending on the particular parameters to be sensed. In the chemical stimulation embodiment, the catheter 50 may be implanted in the hypothalamus (preferably into the VMH), a cerebral ventricle, the afferents/efferents of the celiac ganglia, or the cervical spinal chord (for introducing the drug into the cerebrospinal fluid).

In the electrical stimulation embodiment, the charge-delivering circuit 86 converts the data in a control signal received from the control device 48 into a stimulation signal delivered to the electrode assembly as a controlled current pulse. At the same time, the charge-balancing active-feedback circuit 74 constantly monitors the actual charge going into and out of the tissue and corrects any mismatch by modifying the input received by the charge delivering circuit 86 from the control device 48, thus constantly and dynamically balancing the charge to minimize or prevent tissue damage.

The sensor 88 (or an electrode functioning as a sensor) detects molecules via physio-chemical reactions (for example biosensors). Some of these molecules are glucose, insulin and leptin, which convey, among other things, information about the energy availability. The information regarding the concentration of these molecules is then converted into an electrical signal which is then delivered to the amplifying and filtering circuit 46, which, in turn, delivers the amplified and filtered information in a feedback signal to the control device 48.

In the chemical stimulation embodiment, the implanted micropump 90 locally delivers a particular drug to the hypothalamus, either directly or indirectly (as described above), for hypothalamic stimulation. For example, BDNF, leptin receptor agonists, orexin receptor antagonists, NPY receptor antagonists, gherelin receptor antagonists, and MC4K/MC3R agonists increase energy expenditure and decrease food intake. Conversely, orexin receptor agonists, leptin receptor antagonists, NPY receptor agonists, gherelin receptor agonists, and MC4R/MC3R antagonists decrease energy expenditure and increase food intake. The micropump 90 can be a piezoelectric-driven micropump, such as the one available from FhG-IFT of Munich. Germany, and it is controlled by the control device 48. An intake end of the micropump 90 is connected to the reservoir 52, which contains a particular drug, and the output end of the micropump 90 is connected to the catheter 50.

As mentioned above, the electrode(s) 32 and/or the conduit 30 or the catheter 50 are implanted within the VMH of the brain. The VMH affects metabolic, reproductive, affective, and locomotor behavior. The VMH can be anatomically divided into four regions that are either not connected or share only very sparse connections. These four regions are the anterior (aVMH), ventrolateral (vIVMH), central (cVMH), and dorsomedial (dmVMH). Stimulation of the VMH increases locomotor activity and nMEE, decreases FIN, promotes lipolysis, and stimulates non-shivering thermogenesis, among other things. In addition, experiments have also shown that VMH activity regulates glucose uptake in skeletal muscles during exercise, and that lesions in the VMH produce obesity and hyperphagia. The activity in the VMH can be influenced by both short and long-term energy availability because it contains numerous leptin receptors, and close to half of its neurons are stimulated by a glucose increase.

Referring again to FIG. 1, the LHA 28 has extensive connections both inside and outside the hypothalamus. It sends and receives projections to and from the cortex, the thalamus, the basal ganglia, the mid-brain, the hippocampal formation, the NTS 21, and most hypothalamic regions. In particular, information from the GI tract reaches the LHA 28 via the NTS 21.

The electrode(s) 32 are implanted in the hypothalamus because VMH activity can directly modulate EE, presumably by up-regulating sympathetic activity and by sustaining it through lipolysis, and VMH activity can indirectly influence FIN. Specifically, the electrodes are preferably implanted in the VMH, and in particular its dorsomedial portion (dmVMH), although implantation into the celiac ganglia may be desired in some instances.

The hypothalamus regulates the energy-homeostasis processes by several mutually interacting hypothalamic nuclei. Within this process, short-term, middle-term, and long-term energy availability are constantly monitored, and FIN and energy expenditure (EE) are consequently adjusted in an attempt to maintain an energy balance and a specific body weight.

As described above, a particular region of the brain, such as the hypothalamic nucleus (particularly the dmVMH), will be electrically stimulated. Neurons exhibit a transient depolarization of the cell membrane caused by ionic currents (action potential) in response to supra-threshold stimulation (i.e., approximately a 20 mV change in the transmembrane voltage). Normally, this transient depolarization is generated as the result of endogenous conditions (i.e. the transmembrane voltage), which are generally induced by naturally occurring ionic (gap junctions) or chemical (synapses) interactions with other cells. However, if a cell is placed in a strong enough electric field (E), the voltage gradient (VF) generated by the field can produce the needed transmembrane voltage to reach threshold, thus artificially triggering an action potential.

The extracellular space surrounding the neurons provides an electrolytic medium, which at low frequencies (<250 MHz) behaves as a conductor, and at frequencies below about 10 MHz behaves with nearly frequency-independent conductivity. It is in this electrolytic medium that the ionic current needed to artificially provoke an action potential (also called a spike) can be generated as a result of extracellular electrical stimulation.

In an electrolytic medium, by contrast to metal conductors, the electrical charge is transported by ions instead of electrons. In particular, in a solution where dissolved ions move in a random fashion, the application of an external electric field can force the ionic movement to align itself with the field. Once the ions are, on average, moving according to the electric field, an electric current is generated. Unlike electrons in metals, ions in solution draw toward them oppositely charged ions and water molecules, forming a sheath around the ion. This sheath, generally referred to as the solvation sheath, “masks” the charge of the ion and effectively reduces it. When the solvent is water, the sheath is called the hydration sheath, and it increases the effective diameter of the ion, thereby increasing, in turn, the drag force experienced by the ion moving in the solution. Since the electric current is a measure of the migration of charge per unit time, a bigger drag force effectively reduces the electric current. In solution, each ion species contributes to the electric current, and this contribution is directly related to the velocity at which each species can move in the solution. Since ions can move in any direction in the solution, their movement, and thus the current, must be treated in vectorial form.

As discussed above, the transmembrane voltage needs to be sufficiently increased to artificially trigger an action potential. In order to artificially increase the transmembrane voltage, an external current must be supplied to the solution. This can be achieved by placing electrodes in the solution. At low frequencies and beyond a certain distance from the electrode, the voltage drops according to Ohm's law. The voltage drop can be easily calculated if the charge or the current-density distribution is known.

While the electric current on the electrodes (metal most of the time) is composed of electrons, in the tissue it is composed of ions. Therefore, a charge-carrier exchange must occur at the electrode-tissue interface. This exchange can take place through two different pathways, one through capacitive coupling and the second one through a variable resistive channel involving electrochemical reactions between the electrode and the solution in the tissue. These electrochemical reactions can be reversible or irreversible. Irreversible reactions will erode the electrode and deposit electrode material into the tissue, causing damage.

Also, irreversible water reactions will generate oxygen gas and hydrogen ions at the cathode, and hydrogen gas and OH— ions at the anode, decreasing and increasing the pH respectably. Charge injection through the capacitive pathway is known as capacitive current, and charge injection through the resistive pathway is known as faradaic current. The extent to which one pathway dominates the charge injection, as well as to what extent the faradaic reactions are reversible, is highly dependant on the material that makes up the electrode and on the voltage at the electrode.

Depending on the materials that make up the electrode, a DC equilibrium voltage known as the half-cell potential will be generated. When a voltage is established between the electrode and the tissue (which, due to the half-cell potential, occurs as soon as the electrode is in contact with the tissue), oppositely charged ions move closer to the electrode surface and generate a double-layer capacitor that behaves similarly to a parallel plate capacitor. The double-layer capacitor has a large effect on the voltage gradient, which experiences a nearly exponential fall across this double layer. In order to estimate the thickness of this double-layer capacitor, attention should be paid to the ionic distribution under the presence of an electric field. For bipolar electrodes, the voltage drops rapidly when moving away from the electrode. The fact that the voltage drop is so pronounced, together with the fact that higher voltages can lead to irreversible reactions, severely limits the radius of influence from an electrode.

As discussed previously, there are two pathways through which charge can be injected into the tissue: capacitive current (i_{c}) and a faradaic current (i_{f}). A simple electrical model of an electrode immersed in an electrolyte (e.g., tissue) can be constructed using these two charge-injection pathways and the electrode half-cell potential. At equilibrium conditions, and with no current flowing, the electrode voltage (or electrode potential) is the half-cell potential (Φ_{HC}), and the departure from this potential is called the overpotential.

Since any measurement involves at least two electrodes, in reality, any measurement would involve the Φ_{HC} of both electrodes, and therefore the Φ_{HC} of a single electrode cannot be measured. When the overpotential is low, the electron transfer process (i.e., chemical reactions) is restricted, thus rendering an extremely high impedance through the faradaic (Z_{F}) pathway; consequently most of the charge is injected through i_{c}.

An empirical relationship has been established between charge/phase and charge density/phase for determining the damage threshold for tissues and electrodes. The electrode(s) can be damaged, for example, by corrosion that occurs during the anodic phase of the stimulation, which is when metal can be oxidized. In order to avoid corrosion, the net charge injected should be zero. As a consequence, a charge-balanced pulse should be used, and the anodic amplitude should be restricted to the reversible region.

There are two types of tissue damage that can occur. The first type of damage is due to the production of toxic reactions at an intolerable rate, which could include a local change in pH. Fortunately, a charge-balanced pulse can restrict the pH shift. The second type of damage is due to the actual neuronal activity or over activity caused by the exogenous current flowing in the tissue.

The charge per phase (i.e., charge per pulse) and the charge density play a determining role in the second type of tissue damage. The charge per phase determines the excitation volume, and the charge density determines the percentage of cells to be depolarized beyond threshold. As the charge per phase increases while maintaining a constant charge density, the pulse duration (i.e., the pulse width) must be increased. A longer pulse width allows diffusion processes to disperse the products of the reactions, thereby limiting reversibility. Increasing the charge density increases the net current and the overpotential, which as explained above, can lead to corrosion and other irreversible reactions during the anodic phase. Since irreversible reactions can occur at high current densities, in order to obtain a safe and effective stimulation, the best combination of current, charge, and charge density should be sought.

As discussed above, the total amount of calories from FIN is either retained, expelled, or expended by the body. In adults, the homeostatic mechanisms of the body tend to balance the FIN and the energy outtake (i.e., energy expelled and expended). The energy that is retained is used for growing. The energy expelled is done so mainly through urine and feces. The energy expended is divided in MEE and nMEE. The nMEE can be further divided into the basal energy expenditure, the thermogenesis due to food consumption, and the energy due to non-mechanical activity (e.g., thinking, thermoregulation, etc). The FIN, the energy expelled, and the energy retained are in the form of chemical energy. However, when MEE and nMEE are expended, heat is generally produced.

The MEE can be directly measured by computing the power exerted due to the movements of the test subject, a rat in this case. Power computations can be performed in three ways. First, power can be computed by directly monitoring the triaxial acceleration of the test subject. Second, power can be computed by measuring the triaxial work exerted by the test subject on the floor of a chamber in which the rat is contained. Third, power can be computed by measuring single-axis forces in the vertical direction and calculating the acceleration on the horizontal plane. The force exerted by the test subject on the floor of the chamber is measured using triaxial force transducers. Specifically, four force transducers are used, and the average mechanical power exerted by the test subject is estimated by adding the work done on each force sensor over one second. As described above, the VMH can modulate both the nMEE and, via locomotion, the MEE.

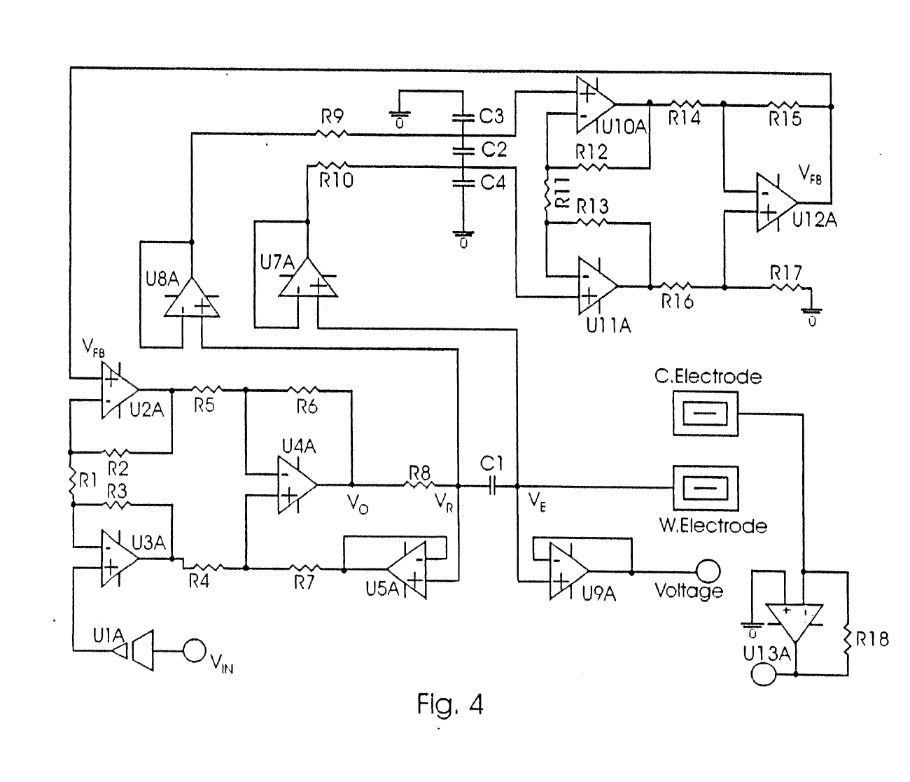

FIG. 4 illustrates an active feedback circuit that automatically balances the injected and extracted charge to avoid damage to the tissue and to the electrode. The circuit can be divided into four functional component groups: (1) an isolation component, (2) a voltage-to-current conversion component, (3) a charge-balance difference measurement component, and, (4) a voltage and current monitoring component.

In order to avoid current paths between the stimulation circuit (including the subject to be stimulated) and ground, the grounds of the circuit and of the laboratory must be isolated from each other. This isolation effectively allows the voltage of the stimulation circuit (including the subject to be stimulated) to “float” with respect to any other instrument in the building, thus providing a secure isolated current path. The isolation component of the circuit is made out of an isolation amplifier (U1A), into which the command signal is delivered (VIN). The output of the isolation amplifier (U1A) provides one of the inputs (positive input) into the voltage-to-current conversion component.

The voltage-to-current conversion is accomplished by forcing the voltage across a resistor (R8) to follow the differential voltage between the inputs of an instrumentation amplifier (U2A, U3A and U4A). By selecting U5A, U7A, U8A, and U9A for operational amplifiers that have a very small input bias current compared to I_{R8}, approximately all of I_{R8} flows through capacitor C₁, and the working and counter electrodes.

At t=0 and V_{IN}=0, there is no charge in C₁ and V_{R}=V_{E}=0. Since the counter electrode is virtually grounded (though U13A), the voltage of both electrodes is forced to be equal and no current flows between them. V_{R} and V_{E} are buffered (U8A and U7A) and then low-pass filtered (R9=R₁₀=R_{LP}, C₃=C₄=C_{LP}, and C₂). The difference between V_{E} and V_{R} is then calculated (i.e., V_{E}−V_{R}) via an instrumentation amplifier (U10A, U11A, and U12A). This instrumentation amplifier is configured in a manner similar to the one composed by U2A, U3A and U4A (R11=∞, R12=R13=R_{B}, and R14=R15=R16=R17, and therefore Vₘ=(V_{E}−V_{R}).

Since VR=VE at t=0, then VFB=0, when V_{IN} changes, Vₒ follows it and I_{R8} starts to flow. As I_{R8} flows, C₁ and the capacitance at the electrode-tissue interface (C_{DL}) begin to accumulate charge (depending on V_{IN}), and V_{R} changes in the same direction as Vₒ. However, as V_{R} changes, V_{O} is compensated by the feedback provided by U5A, and the voltage between Vₒ and V_{R} is forced to remain constant. Since V_{FB} is not affected by fast transient differences between V_{R} and V_{E}, a transient pulse is allowed to go through the electrodes.

When V_{IN} is inverted (to balance the charge), C₁ and C_{DL} are discharged. In the event that the overall charge is not balanced, after one or many cycles, the charge in C₁ does not go to zero, and a DC voltage between V_{E} and V_{R} is established. When a DC voltage between V_{E} and V_{R} exists, V_{FB} changes accordingly, adjusting Vₒ and I_{R8} in order to eliminate the DC voltage between V_{E} and V_{R}, which in turn automatically balances the charge injected and extracted.

In order to verify in real-time what the voltage and the current are between the electrodes, two more components are used. The Op-Amp U9A is configured as a follower, and the voltage at its output is the same as the voltage between the electrodes. As stated before, the counter electrode is virtually grounded by U13A, and the current flowing between the electrodes is forced to flow through R18 (provided that the input bias current of U13A is very low by comparison to the current flowing between the electrodes). Therefore, by measuring the voltage at the output of U13A, the current between the electrodes can be monitored in real-time.

In a particular example of the invention, the electrodes 32 comprised two 50 μm diameter tungsten-microwires (such as CFW-211-022-HML manufactured by California Fine Wire) insulated with a 4 μm thick layer of polyimide, which were passed through a 30-gauge stainless-steel needle. The tips of the wires were longitudinally 1 mm apart from each other. The wires were soldered to a connector (such as MCP-05-SS manufactured by Omnetics), and then the connector, the needle, and the wires were placed into an aluminum mold and dental cement was pored onto the mold to make it a monolithic piece. In both wires, the insulation was removed about 500 μm from the tip to expose an effective area of approximately 25,000 μm² (0.025 mm²). The stimulation was performed in a bipolar configuration between the two microwires.

Thirty six Wistar male rats, weighing between 295 and 340 g (mean 315.26 g) were anesthetized with 2% isoflurane (5% for induction). In order to secure the implant before inserting the electrode, four anchor screws (such as stainless steel 0-80× 3/32 manufactured by Plastics One) were placed around the implantation site. A small hole was drilled, and the electrode was then carefully positioned and inserted into the left dorso-medial portion of the ventromedial hypothalamic nucleus (dmVMH). The target coordinates of the insertion were: anteroposterior: −2.56 mm, mediolateral: 0.5 mm, and ventral, 9.5 mm. The animals where individually housed and food and water were provided ad libitum. After surgery, the animals where allowed a seven-day recovery period and then they were randomly assigned to six groups (n=6): sham (G0), 25 Hz (G1), 50 Hz (G2), 100 Hz (G3), 200 Hz (G4), and 7 kHz (G5).

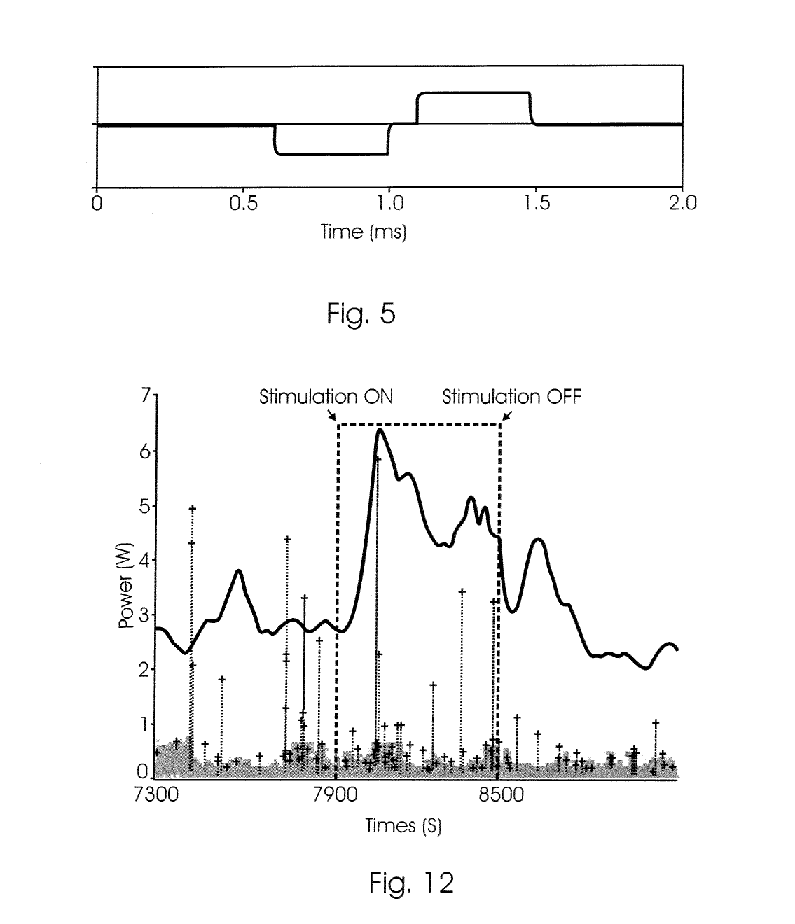

FIG. 5 is a graph illustrating a stimulation waveform where the charge is automatically balanced using the active feedback circuit described above and illustrated in FIG. 4. After the seven-day recovery period, the animals were, one at a time, placed into a metabolic chamber. After a 30-minute familiarization period, a stimulation threshold was established by progressively increasing the starting amplitude (10 μA) by 5 μA increments until a behavioral response was observed. The stimulation consisted of a 30-seconds-ON, 30-seconds-OFF train of 1-ms squared charge-balanced constant-current pulses. The threshold for all animals was between 20 μA and 30 μA. The stimulation frequency was changed according to each animal group. After establishing the stimulation threshold, the animals were returned to their regular cages. One day after establishing the stimulation threshold, the animals were again placed into the metabolic chamber. Following a 30-minute period to record the baseline for the nMEE, the rats were stimulated (at the threshold intensity) for 90 minutes. Following the stimulation, a resting period of 40 minutes was recorded. While the rats were in the chamber, access to food was denied in order to avoid any metabolic responses due to food intake (i.e., thermogenic effect).

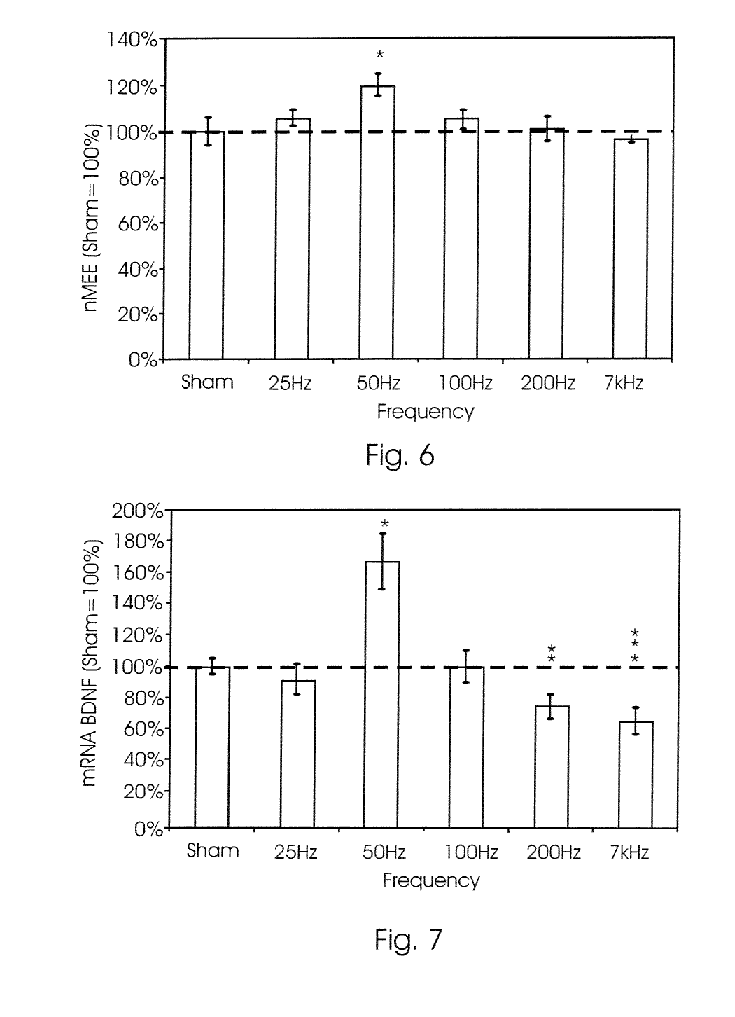

FIG. 6 is a bar graph illustrating the effect of stimulation frequency on nMEE. Overall, stimulation frequency has an effect on the nMEE (ANOVAp=0.022); however, the nMME does not respond significantly to all stimulation frequencies. Although the nMEE was increased for all but the highest frequency tested (7 KHz), at which it showed a decreasing trend, it was only at 50 Hz that the change was statistically significant (p<0.05) when compared to the sham group. FIG. 7 summarizes the results of the stimulation at different frequencies. The concentration of BDNF mRNA in the hippocampus was significantly increased by 50 Hz stimulation and showed a decreasing tendency as the frequency increased above 100 Hz.

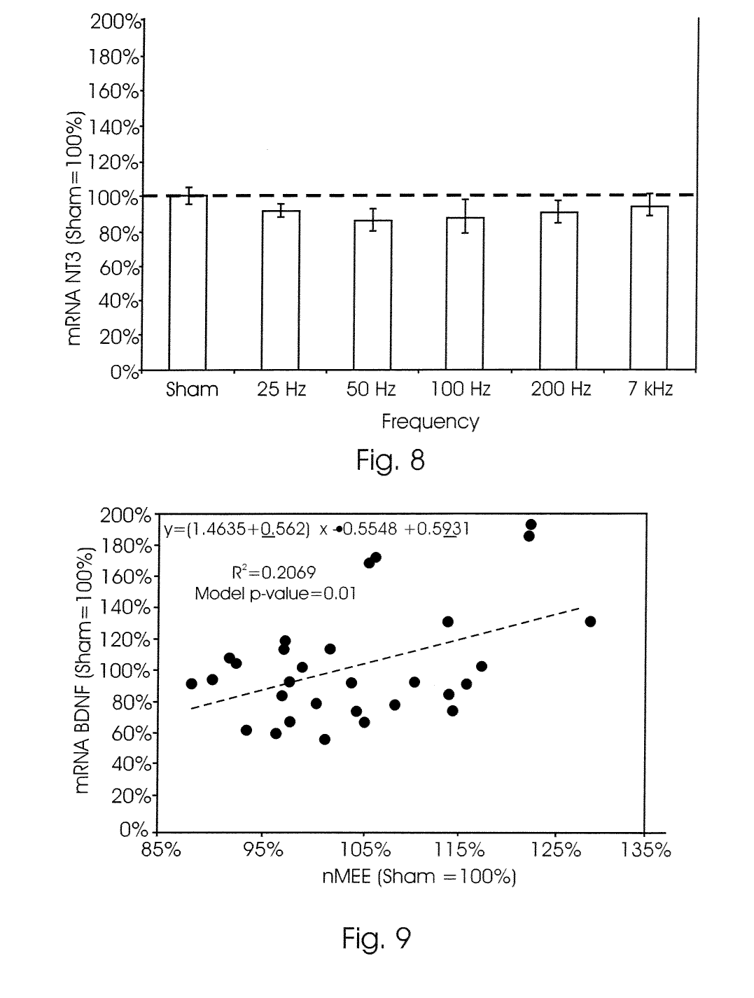

With the purpose of using it as a control for the BDNF mRNA, the concentration of neurotrophic factor 3 (NT3) mRNA in the hippocampus was measured. NT3 mRNA was not significantly altered by hypothalamic stimulation at any frequency (ANOVAp=0.6984) as shown in FIG. 8.

Referring again to FIG. 6, the stimulation frequency significantly influences the nMEE response. The data suggests that although frequencies between 25 and 200 Hz have a tendency to increase the nMEE, at the levels of stimulation used, this tendency was only statistically significant at 50 Hz. If the stimulation amplitude had been altered, then the effects on the nMEE would have simply reflected the number of cells that would have been recruited.

As shown in FIG. 7, the BDNF mRNA responded in an unexpected way to an acute 90-minute dmVMH stimulation. The response suggests a strong frequency dependency with qualitative changes: a 68% increase at 50 Hz, a 24%, and a 33% decrease at 200 Hz and at 7 KHz, respectively. This suggests that activity at least certain hippocampal cells are depends on the frequency of the stimulation signal delivered to the hypothalamus. Since a spread in the actual stimulation current is a remote possibility due to both the electrical isolation (all the stimulation current is returning through the counter electrode) and to the bipolar nature of the stimulation, a direct or indirect neuronal pathway is implied. In addition the fact that dmVMH stimulation at 7 KHz (i.e., a frequency at which axonal action potentials are blocked) significantly decreases hippocampal BDNF mRNA in the hippocampus, further emphasizes a direct or indirect neuronal connection. In addition, NT3 mRNA, which is up-regulated by mechanisms that differ from those that up-regulate BDNF, was used as a control for BDNF mRNA. The fact that dmVMH stimulation affects BDNF mRNA but does not affect NT3 mRNA, suggests that VMH stimulation specifically affects BDNF mRNA and not other neurotrophic factors.

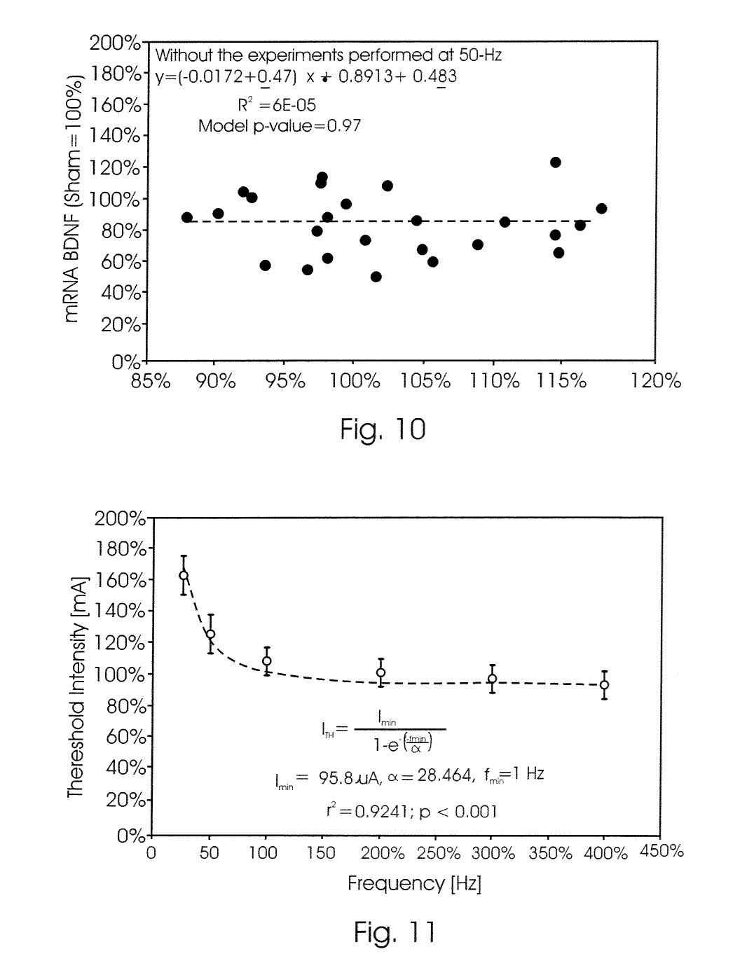

A regression analysis performed between the hippocampal BDNF mRNA and the nMEE suggest that although qualitatively similar responses at 50 Hz and 7 KHz are observed, there is no causal relationship between them. FIG. 9 illustrates that a mild but statistically significant correlation exists between the BDNF and nMEE. However, FIG. 10 shows that this correlation completely disappears when the animals in the 50-Hz group are removed. This observation suggests that there is no causal relationship and that the pathways responsible for both of these effects (i.e., nMEE and BDNF mRNA responses) are different. Both, however, might be activated by similar mechanisms in response to VMH stimulation.

The motivation behind the escape-response threshold study was to characterize a potentially undesired effect for a therapy that would be using electrical stimulation in the dmVMH. The results, summarized in FIG. 11, show that the threshold to elicit an escape-response decreases as the frequency increases. This phenomenon is likely to be caused by a temporal summation effect, in which a higher release by the presynaptic terminals causes a greater response at the postsynaptic terminals due to temporal overlap in the excitatory postsynaptic evoked potentials (EPSP). This escape response can be characterized as the flight portion of the fight-or-flight response, which is elicited through activation of the sympathetic nervous system (SNS). In particular, the fight-or-flight response is mediated by the release of catecholamines (i.e., epinephrine and to a lesser degree norepinephrine) from the adrenal gland, which is one of the target structures of the VMH-splanchnic-nerve pathway. The minimum current necessary to elicit the escape response (Imin) is, on average, about three times the current required to cause a significant increase in nMME. Therefore, by characterizing the threshold of the escape response, which can be regarded as an undesirable side effect, the current intensity that should not be exceeded in a protocol where VMH stimulation is used was identified.

The dependence of the NMEE, the hippocampal BDNF mRNA, and the threshold of the escape response, on the stimulation frequency in the dmVMH was investigated. The results show that nMEE can be more effectively increased when dmVMH stimulation is delivered at 50 Hz, and that a marginal decreasing trend occurs at a frequency that blocks axonal conduction (i.e., 7 KHz). These findings parallel the results obtained for hippocampal BDNF mRNA. However, a regression analysis between nMEE and hippocampal BDNF mRNA suggests that a causal relationship between them is not a plausible mechanism of action. On the contrary, the data suggest that both effects might be caused by activating parallel pathways operating through different mechanisms. In order to elucidate the extent of the dmVMH-stimulation effect on the neurotrophins in the hippocampus, the NT3 response was investigated. The fact that the hippocampal NT3 mRNA is not affected by dmVMH stimulation suggests that dmVMH stimulation specifically affects the BDNF-mRNA concentration in the hippocampus.

To determine the maximum current to be used in a dmVMH-stimulation-protocol the threshold to elicit an escape response was investigated. The results showed that the current necessary to elicit an escape response is, on average, three times greater than that required to significantly affect both the nMEE and the hippocampal BDNF mRNA.

FIG. 12 illustrates the VMH stimulation effect on TEE. The TEE response to VMH stimulation happens a few seconds after the stimulation on set (approximately 20 seconds). The delay in the TEE due to the traveling time of the gas to arrive at the analyzers was considered in order to align the stimulation onset with the TEE. For the particular experiment from which this figure was derived, stimulation consisted of a 10-minute pulse train with an amplitude of 40 μA, a frequency of 50 Hz, and a pulse-width of 100 μs.

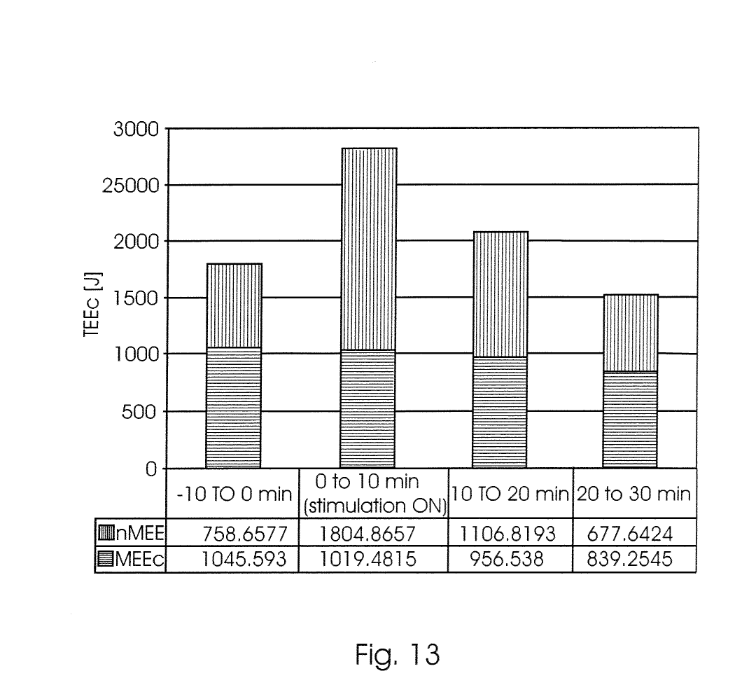

FIG. 13 also illustrates a VMH stimulation effect on the TEE. Cumulative energy is shown in 10-minute bins starting 10 minutes before stimulation was started and ending 20 minutes after stimulation was stopped. The TEEC is shown as the addition of the MEEC and the nMEE. As opposed to FIG. 28, the cumulative energy is shown and not the power.

Although the present invention has been described with reference to specific embodiments, these embodiments are illustrative only and not limiting. Many other applications and embodiments of the present invention will be apparent in light of this disclosure and the following claims. In addition, as mentioned above, although the present invention has been described in connection with testing and experiments on rats, those skilled in the art will recognize that the principles and teachings described herein may be applied to other mammalian species, including humans.

1. A method for modulating a brain function selected from the group consisting of at least one of energy expenditure regulation and the expression of brain-derived neurotrophic factor (BDNF) in the brain of a subject, the method comprising the steps of:
generating a stimulation pattern from a predetermined set of stimulation parameters;
converting the stimulation pattern into a stimulation signal, and
delivering the stimulation signal to a selected part of the brain so as to modulate the brain function.

2. The method of claim 1, wherein the first step in the method is the step of implanting a stimulation signal delivery mechanism in the selected part of the brain, and wherein the step of delivering the stimulation signal is performed by the stimulation signal delivery mechanism.

3. The method of claim 1, further comprise the steps of:
generating a feedback signal from a sensor, wherein the feedback signal represents the value of a measured parameter; and
adjusting the stimulation parameters in response to the feedback signal.

4. The method of claim 1, wherein the stimulation signal is an electrical stimulation signal delivered to a part of the brain selected from the group consisting of the hypothalamus and the VMH-splanchnic pathway.

5. The method of claim 1, wherein the stimulation signal is a chemical stimulation signal delivered to the hypothalamus by means of a dosage regimen of an appropriate chemical.

6. The method of claim 5, wherein the chemical stimulation signal is delivered indirectly to the hypothalamus via at least one of a cerebral ventricle, the cerebrospinal fluid and the blood circulation.

7. The method of claim 1, wherein the steps of generating a stimulation pattern and converting the stimulation pattern into a stimulation signal are performed with a control device selected from the group consisting of a microprocessor, a microcontroller, and a state machine.

8. The method of claim 3, wherein the feedback signal is generated by at least one of a brain-implanted sensor and a non-invasive sensing device.

9. The method of claim 2, wherein the stimulation signal is an electrical stimulation signal, and wherein the stimulation signal delivery mechanism comprises a brain-implanted electrode.

10. The method of claim 2, wherein the stimulation signal is a chemical stimulation signal in the form of a drug dosage regimen, and wherein the stimulation delivery mechanism comprises a pump that delivers the drug dosage regimen to the selected part of the brain through a conduit implanted in the selected part of the brain.

11. The method of claim 4, wherein the stimulation parameters are selected from the group consisting of electrical current intensity, pulse width, pulse frequency, wave shape, duration of stimulation, and the repetition of stimulation.

12. The method of claim 5, wherein the stimulation parameters are selected from the group consisting of drug type, drug flow rate, total delivered drug volume per stimulation session, and repetition rate of drug delivery.

13. The method of claim 6, wherein the stimulation parameters are selected from the group consisting of drug type, drug flow rate, total delivered drug volume per stimulation session, and repetition rate of drug delivery.

14. The method of claim 5, wherein the chemical stimulation signal is provided by a chemical selected from the group consisting of at least one of BDNF, leptin receptor agonists, orexin receptor antagonists, NPY receptor antagonists, gherelin receptor antagonists, and MC4R/MC3R agonists.

15. The method of claim 6, wherein the chemical stimulation signal is provided by a chemical selected from the group consisting of at least one of BDNF, leptin receptor agonists, orexin receptor antagonists, NPY receptor antagonists, gherelin receptor antagonists, and MC4R/MC3R agonists.

16. A system for modulating a brain function selected from the group consisting of at least one of energy expenditure regulation and the expression of brain-derived neurotrophic factor (BDNF) in the brain of a subject, the system comprising:
a control device operable to generate a stimulation pattern from a predetermined set of stimulation parameters, and to convert the stimulation pattern into a stimulation signal; and
a stimulation signal delivery mechanism, configured for implantation into a selected part of the brain, that receives the stimulation signal from the control device and delivers the stimulation signal to the selected part of the brain.

17. The system of claim 16, further comprising a sensor that generates a feedback signal in response to measured parameters affected by the stimulation signal, whereby the control device is operable to receive the feedback signal and to adjust the stimulation parameters in response thereto.

18. The system of claim 16, wherein the stimulation signal is an electrical signal, and wherein the stimulation signal delivery mechanism includes a brain-implantable electrode.

19. The system of claim 16, wherein the stimulation signal is a chemical signal in the form of a drug dosage regimen, and wherein the stimulation signal delivery mechanism includes a brain-implantable conduit and a brain-implantable micropump, wherein the control device delivers a control signal to the micropump, and wherein the micropump delivers the drug dosage regimen to the conduit in response to the control signal.

20. The system of claim 16, wherein the control device is selected from the group consisting of a microprocessor, a microcontroller, and a state machine.

21. The system of claim 17, wherein the stimulation parameters are selected from the group consisting of electrical current intensity, pulse width, pulse frequency, wave shape, duration of stimulation, and the repetition of stimulation.

22. The system of claim 19, wherein the stimulation parameters are selected from the group consisting of drug type, drug flow rate, total delivered drug volume per stimulation session, and repetition rate of drug delivery.

23. The system of claim 19, wherein the chemical stimulation signal is provided by a chemical selected from the group consisting of at least one of BDNF, leptin receptor agonists, orexin receptor antagonists, NPY receptor antagonists, gherelin receptor antagonists, and MC4R/MC3R agonists.

24. A method of modulating brain-derived neurotrophic factor (BDNF) expressed in the brain of a subject, the method comprising the stimulation of a part of the brain selected from the group consisting of at least one of the hypothalamus and the VMH-splanchnic pathway so as to modulate the expression of BDNF.

25. The method of claim 24, wherein the stimulation is performed by the delivery of at least one of an electrical stimulation signal and a chemical stimulation signal to the selected part of the brain.
